(19) United States
(12) Patent Application Publication
(10) Pub. No.: US 2006/0186358 A1
(43) Pub. Date: Aug. 24, 2006

(54) Shopping cart sanitizing, disinfecting and cleaning system and method of use

(76) Inventor: Will Couvillion, Pueblo, CO (US)

Correspondence Address: Jones Waldo Holbrook & McDonugh, Suite 1500, 170 South Main Street, Salt Lake City, UT, 84101-1644, US

(21) Appl. No.: 11/339,221

(22) Filed: Jan. 25, 2006

Related U.S. Application Data
(60) Provisional application No. 60/646,730, filed on Jan. 25, 2005.

(51) Int. Cl. A61L 2/10; A61L 2/00

(52) U.S. Cl. 250/504 R; 422/24

(57) ABSTRACT

A system and method for substantially disinfecting a cart against one or more contaminants. The system includes an enclosure, adapted to receive a cart; and a UV light source, coupled to the enclosure. The enclosure can be a free-standing enclosure, or it can be integrated into a housing structure. In one embodiment the UV light source is a cold cathode lamp. The light source can be powered through a conventional power source, such as an electrical outlet, or it could be hard-wired into the housing structure, or could even be powered by a battery. In other embodiments, the UV light source further includes a reflector and a switch coupled between the power source and the UV light source. The method includes providing an enclosure, adapted to receive the cart, and having a UV light source, coupled thereto. The cart is then inserted into the enclosure, and irradiated with light emitted from the UV light source until the desired germicidal/fungicidal effect is obtained. The cart can then be removed from the enclosure substantially disinfected against the one or more contaminants.

### STATEMENT CONCERNING PRIORITY

This application claims priority to Application Ser. No. 60/646,730, filed on Jan. 25, 2005.

### BACKGROUND

As has recently been a topic of considerable discussion in the news, shopping carts are a veritable playground for germs, contaminants and all sorts of communicable diseases. In an effort to resolve this concern, some retail outfits have begun hand cleaning their carts regularly. However, such an approach is not effective for the following reasons:

1) the chemicals used to clean the carts can, over time corrode the materials from which the carts are made;

2) it takes a significant devotion of human resources to do this job, and therefore, the process is inefficient;

3) some types of cleaners may not be effective against some types of contaminants; and

4) some types of chemical cleaners may cause allergic reactions in customers and employees that, in some instances are worse than the diseases being combated.

Just by virtue of their use by multiple people (some with better personal hygiene than others) shopping/food carts are contaminated with all sorts of bacteria, viruses, and in some instances fungi. Additionally, juices from raw meats and other sources can also transmit disease to people simply through their use of a contaminated cart.

In short, shopping carts present a significant health risk to both employees in the retail sector, as well as to the consuming public at large. Therefore, a system that is able to quickly, cheaply and effectively rid carts of a wide array of contaminants would be desirable.

### SUMMARY

The invention in its various embodiments as taught herein is a system for substantially disinfecting a cart against one or more contaminants. The system includes an enclosure, adapted to receive a cart; and a UV light source, coupled to the enclosure. The enclosure can be a free-standing enclosure, or it can be integrated into a housing structure. In one embodiment the UV light source is a cold cathode lamp. The light source can be powered through a conventional power source, such as an electrical outlet, or it could be hard-wired into the housing structure, or could even be powered by a battery. In other embodiments, the UV light source further includes a reflector and a switch coupled between the power source and the UV light source.

A method of substantially disinfecting a cart against one or more contaminants is also disclosed. One step in the method is providing an enclosure, adapted to receive the cart, and having a UV light source, coupled thereto. The cart is then inserted into the enclosure, and irradiated with light emitted from the UV light source until the desired germicidal/fungicidal effect is obtained. The cart can then be removed from the enclosure substantially disinfected against the one or more contaminants.

### BRIEF DESCRIPTION OF THE DRAWINGS

FIG. 1 is a side perspective view of a cart sanitizing system according to one embodiment of the present invention.

FIG. 2 is a schematic drawing of a structure having an integrated cart sanitizing system according to yet another embodiment of the present invention.

FIG. 3 is a front view of a cart sanitizing system according to one embodiment of the present invention.

FIG. 4 is a front view of a cart sanitizing system according to yet another embodiment of the present invention.

FIG. 5 depicts an example of a light source that is suitable for use in connection with the present invention.

FIG. 6 illustrates one embodiment of a frame structure suitable for use with the present invention.

FIG. 7 depicts a front view of a cart sanitizing system according to yet another embodiment of the present invention.

FIG. 8 is side view of a cart enclosure according to one embodiment of the present invention.

FIG. 9 is a front view of a cart sanitizing system according to yet another embodiment of the present invention.

FIG. 10 shows another side view of a cart enclosure according to one embodiment of the present invention.

FIG. 11 shows a top view of a light source according to one embodiment of the present invention.

FIG. 12 shows a front view of a cart sanitizing system according to yet another embodiment of the present invention.

FIG. 13 shows a front/side view of a cart sanitizing system according to yet another embodiment of the present invention.

### DETAILED DESCRIPTION OF THE DRAWINGS

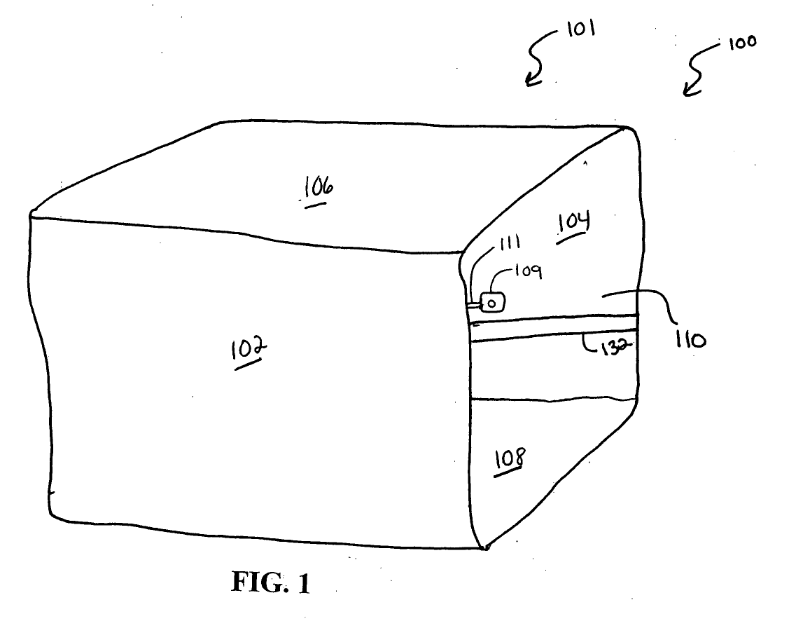

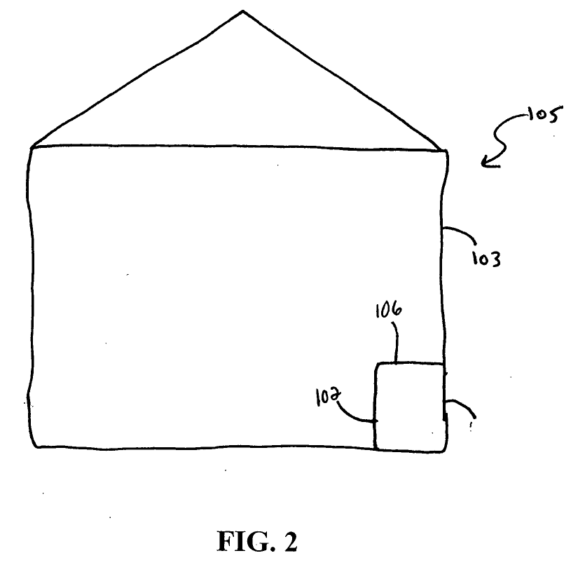

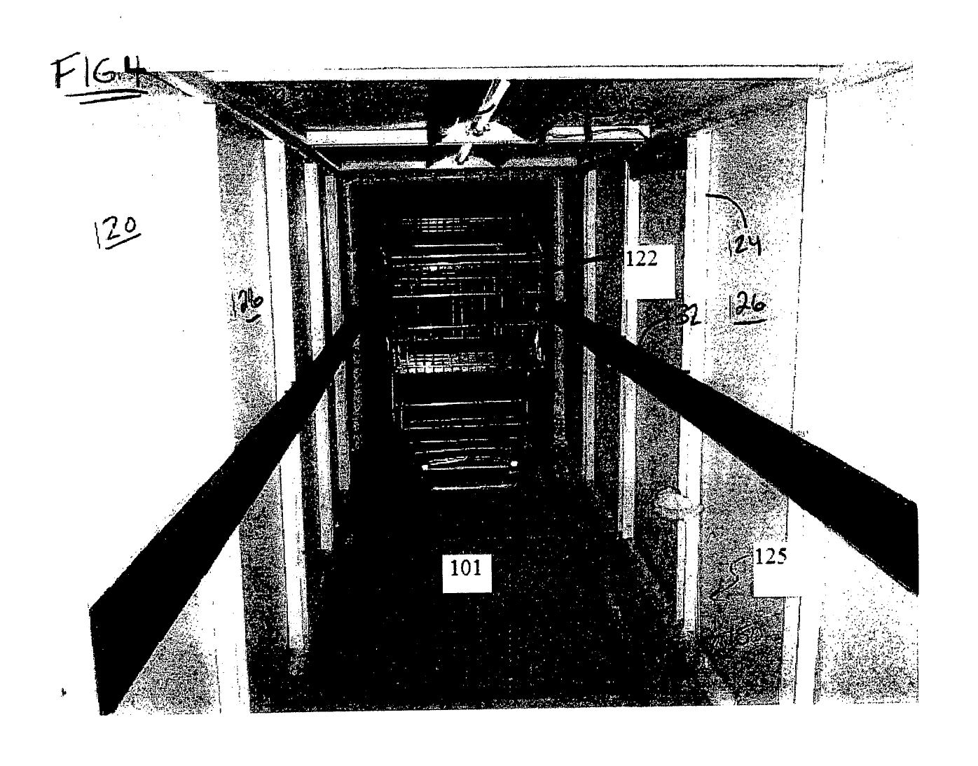

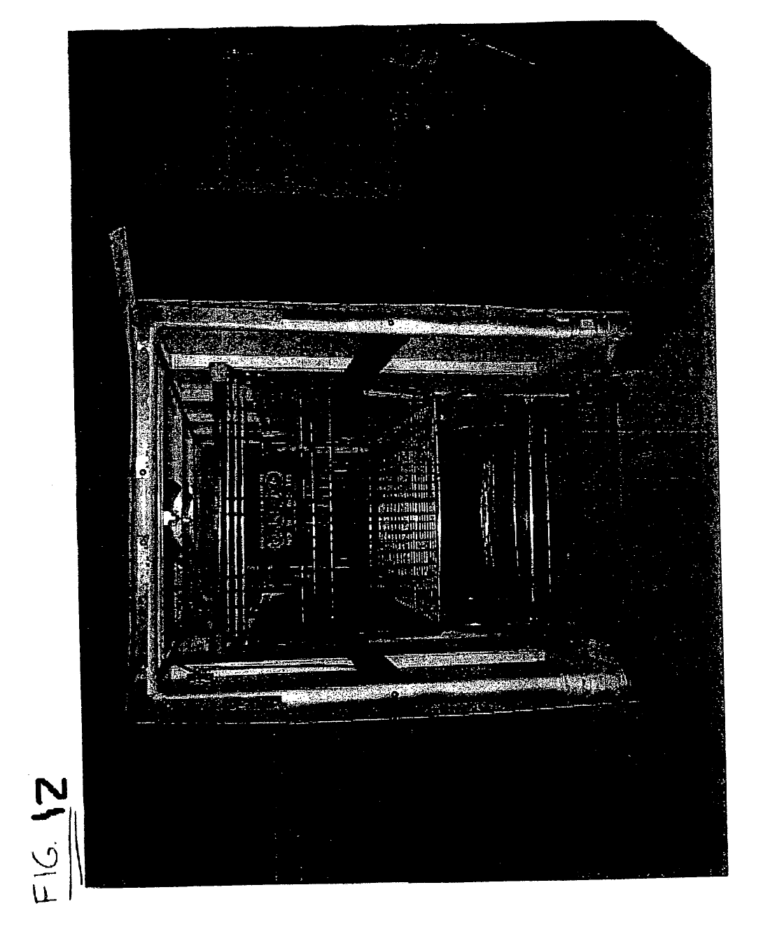

In FIG. 1 is shown a side perspective view of a cart sanitizing system 100 according to one embodiment of the present invention. The system 100 includes an enclosure 101. The enclosure 101 is made up of a first and second side wall 102, 104, a top 106 and an optional bottom surface 108 upon which a cart 122 (FIGS. 4, 8, 12 and 13) is placed while being sanitized. It is noted that the enclosure 101 as shown in the present figures is depicted as a free-standing piece. However, integrated enclosures are also considered to fall within the scope of the present invention. For example, as depicted in FIG. 2, in one embodiment of the present invention, one side 102 of the enclosure 101 is a panel, which is coupled to the top 106, which is turn coupled to a wall 103 of the structure 105 in which the system 20 is housed (e.g. the store wall, etc.). Thus, the structure wall 103 serves as the second side wall 104 of the enclosure 101.

As can also be appreciated, the structure 105 may also be constructed in such a manner that it serves as both side walls 102, 104, the top 106, the bottom 108, or any combination thereof. In short, the enclosure 101 could be defined in part, or in whole by the structure 105. Moreover, a bottom surface 108 may not be necessary as the surface upon which the enclosure 101 rests could also serve as the bottom surface 108.

The enclosure 101 includes an opening 110 that is adapted to receive a cart 122. It is noted that the term “cart” includes, but is not limited to, shopping carts, warehouse carts, lumber carts and luggage carts, and each of these vehicle types may be sanitized by the present system. Of course, as would be apparent to one skilled in the art, the size of the enclosure could be varied according to the specific dimensions and/or number of the cart(s) being sanitized. It is also noted that while the present disclosure refers primarily to its use in connection with “carts,” it is also contemplated to be suitable for use with baskets and other types of shopping devices that are used to transport items, and that are subject to contamination.

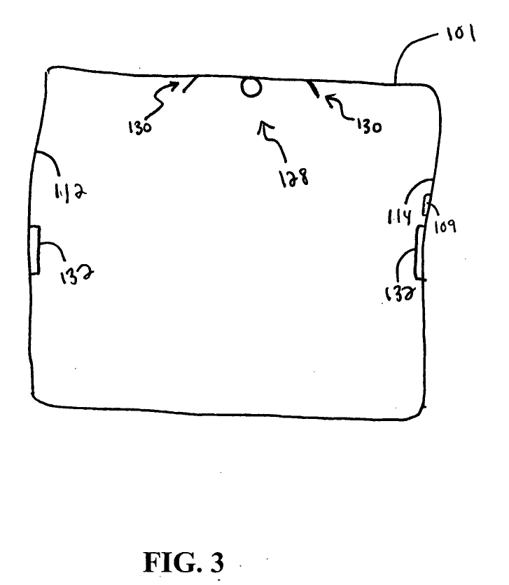

As better seen in FIG. 3, coupled directly to, or proximate to, the top inside of the enclosure 101 is a UV light source 128. In the present embodiment, the UV light source 128 is a lamp and also has parabolic reflectors 130 disposed at approximately 45 degree angles on each side. These reflectors 130 help contain the UV light emitted by the light source 128 in the enclosure 101, and direct the UV light downward toward the carts 122. By including reflectors 130 of this sort, the UV light is more effectively targeted toward the shopping carts 122, and the efficiency of the system 100 is thus improved.

It is noted that the efficiency of the present system 100 can further be improved by simply painting the inside surface of 112, 114 of the side walls 102, 104 white (or alternatively, purchasing side paneling material that is already white). The white inside surfaces 112, 114 also reflect the UV light to some extent, thus directing more of it toward the shopping carts 122, and further minimizing the amount of bleeding light.

The UV light source 128 is the means by which the contaminants on the cart 122 are eliminated. Specifically, when the carts 122 are placed in the enclosure 101 through opening 110, they can then be irradiated by the UV light source 128. The UV light source 128 emits a wavelength, or a range of wavelengths that are germicidal or fungicidal in their effect. Thus, by subjecting a cart 122 to the UV light as emitted by the light source 128, the cart 122 becomes sanitized without the necessity of harsh chemicals, or lengthy hand cleaning processes.

It is noted that the amount of irradiating time necessary in order to achieve the desired germicidal or fungicidal effect depends on a number of factors including: 1) the type of contaminant to be eliminated; 2) the type of lamp being used; 3) the amount of energy being supplied to the lamp, and the resultant wavelengths of light being emitted; and 4) the proximity of the lamp to the contaminant. However, the calculation of the amount of time necessary to achieve the desired germicidal/fungicidal effect based on these factors could be readily done without undue experimentation by one of ordinary skill in the art.

It is also noted that presently it is believed that wavelengths of 185 nm-254 nm are most effective at killing single cell microorganisms (these wavelengths are sometimes referred to as “germicidal UV” or “UVC”). Thus, lamps emitting those wavelengths are considered to be suitable for use with the present invention. However the invention is not intended to be limited to use with any particular UV lamp or any particular wavelength.

Another feature of the embodiment of FIGS. 1 and 3 is the inclusion of side bumper guards 132 coupled to the side walls 102, 104 of the enclosure 101. The bumper guards 132 help direct the shopping cart 122 away from the side walls 102, 104 thus avoiding collisions that could damage the enclosure 101.

FIG. 4 shows yet another embodiment of the present system 100. This embodiment includes a framing component 125, which is standard aluminum framing 124, coupled to ABS tube framing 150. The combination of aluminum 124 and ABS 150 framing is advantageous because of it affords both light weight and durability. However, other types of framing would also be suitable for use with the present invention including, but not limited to, wood, PVC and steel.

Attached to the framing 125 are side panels 126 and one or more top panels 129. In the present embodiment, the side and top panels 126, 129 are fiberglass reinforced plastic (FRP). Other suitable materials for the side and top panels 126, 129 include, but are not limited to ABS standard sized top and side panels with the UV (ultra-violet) lamps pre-installed.

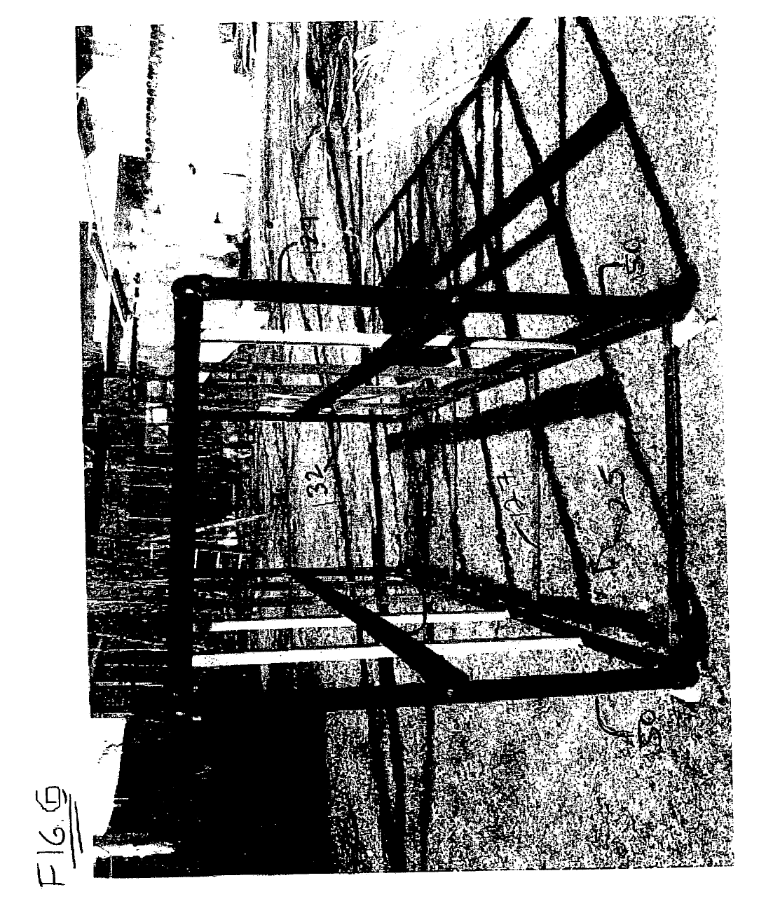

Together, the top and side panels 126, 129 define the enclosure 101. As was previously noted, it is into this enclosure 101 that a shopping cart 122 is placed. As shown in FIG. 4, the side bumper guards 132 are attached at about mid-way up the side walls of the enclosure 101. As is seen in FIG. 6, the bumper guards 132 in one embodiment can be attached directly to the framing 125.

FIG. 4 better shows the parabolic reflectors 130 adjacent the UV light source 128. As noted previously, these reflectors 130 help contain the UV light emitted by the lamp 128 in the enclosure 101, and direct the UV light downward toward the carts 122. By including reflectors 130 of this sort, the UV light is more effectively targeted toward the shopping carts 122, and the efficiency of the system 120 is thus improved.

The embodiment of FIG. 4 also includes white inside surfaces 126, 129. As noted above, the white inside surfaces 126, 129 also reflect the UV light to some extent, thus directing more of it toward the shopping carts 122, and further minimizing the amount of bleeding light.

Reflectors 130 in the present embodiment are polished aluminum. However, other suitable reflector materials include, but are not limited to, highly polished parabolic aluminum and chrome.

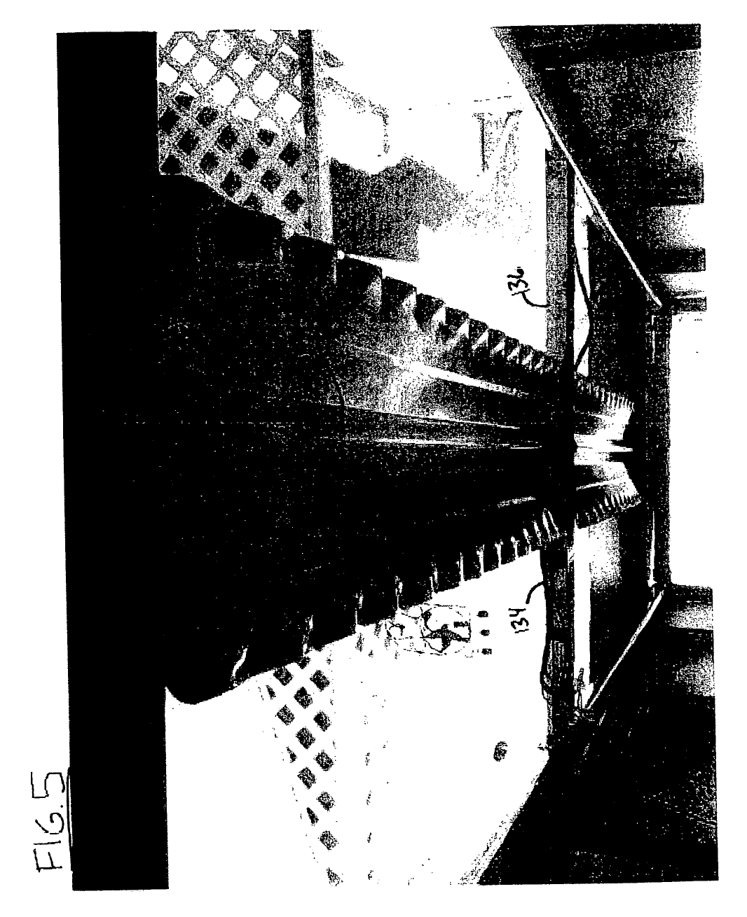

As better seen in FIG. 5, in the present embodiment, the UV light source 128 can be a cold cathode lamp powered by a conventional power source 134. The light source 128 and reflectors 130 are supported by a top framing piece 136 that is coupled on each end with the side ABS framing 150.

The cold cathode lamps used in the present embodiment are available from Universal Light Source, of San Francisco, Calif. Cold cathode lamps are advantageous because they last up three times longer than other types of UV light sources. Additionally, cold cathode lamps do not create large amounts of heat, which can create unpleasant surroundings, or, in some instances, can be hazardous.

However, numerous other UV light sources would also be suitable for use with the present invention.

In one embodiment, the UV lamp 128 is encased in quartz glass, which can provide even greater durability and longevity.

FIG. 6 shows a cart sanitizing system under construction, revealing in greater detail the framing system 125. It is also noted that in this embodiment, the bumper guards 132 are coupled to steel tube framing 150. The framing 125 can also include bottom framing components 127 to further support the system 120.

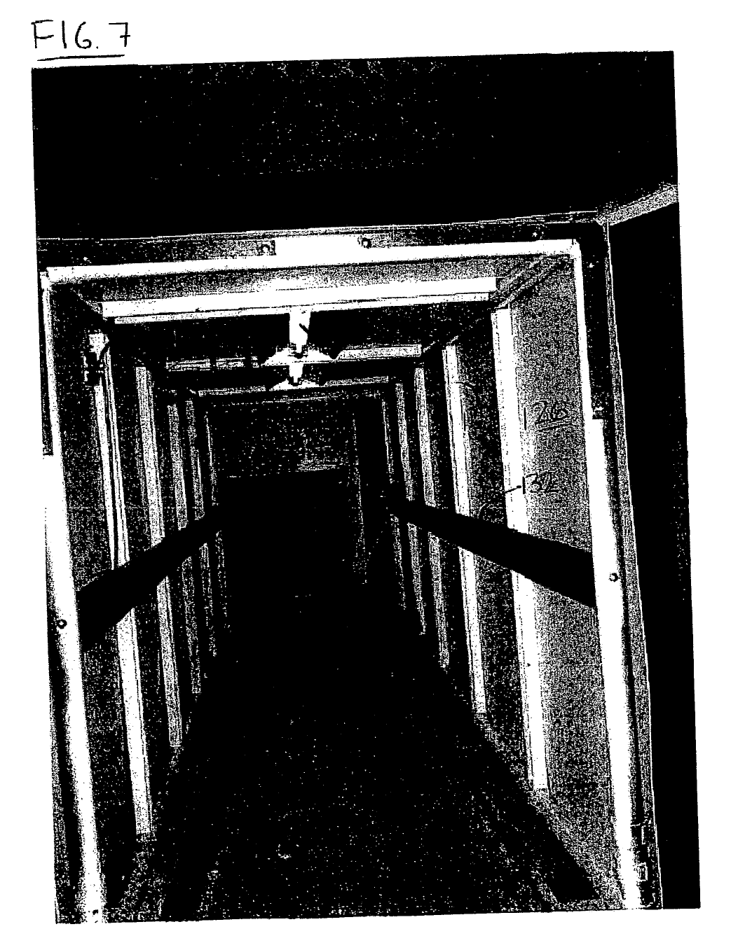

FIG. 7 shows an inside front view of yet another embodiment of the present invention.

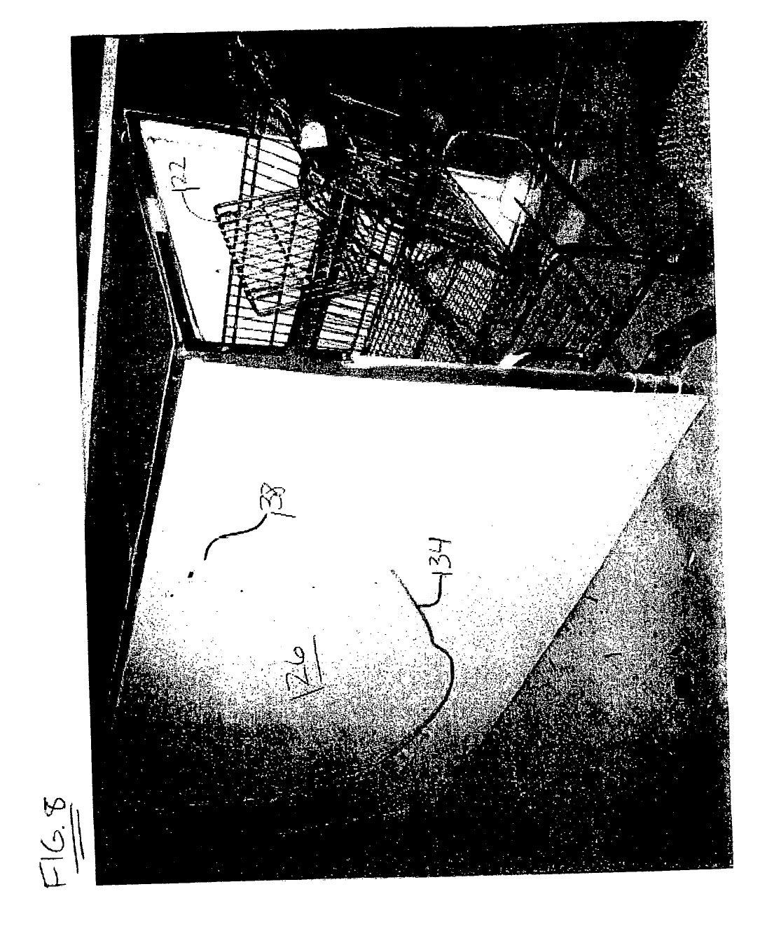

FIG. 8 provides an outside view of one embodiment of the present system 120 that includes a switch 138 coupled to the UV light source 128. Thus, a user can easily turn the UV light source 128 on and off from safely outside the enclosure 101. Also seen in FIG. 8 are rails 140. The rails 140 help direct the cart wheels 142, and thus the cart 122, into the enclosure 101 properly. The inclusion of rails 140 makes putting multiple carts 122 into the enclosure 101 easier, and inasmuch as the rails 140 line up the carts 122 to some extent, the carts 122 are more inclined to properly stack—thus permitting more carts 122 to be placed in the enclosure 101.

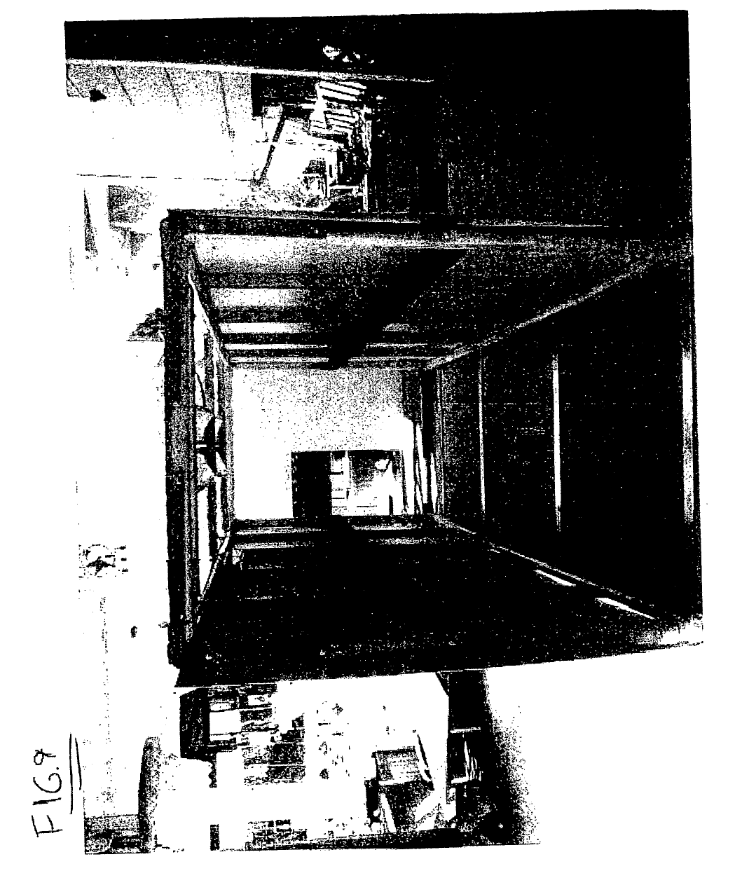

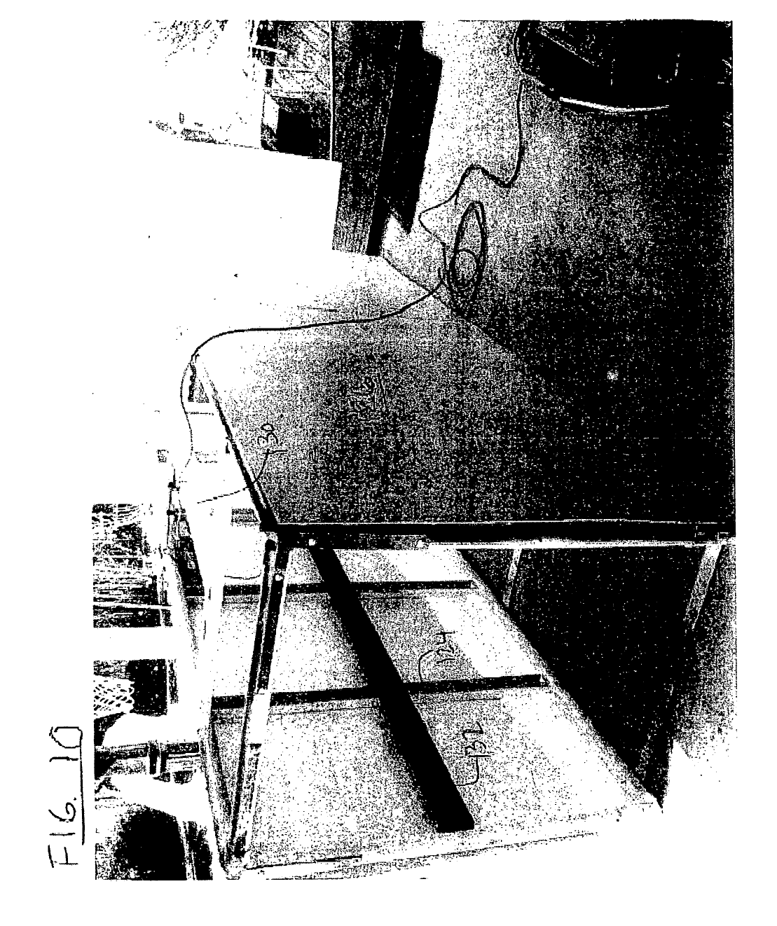

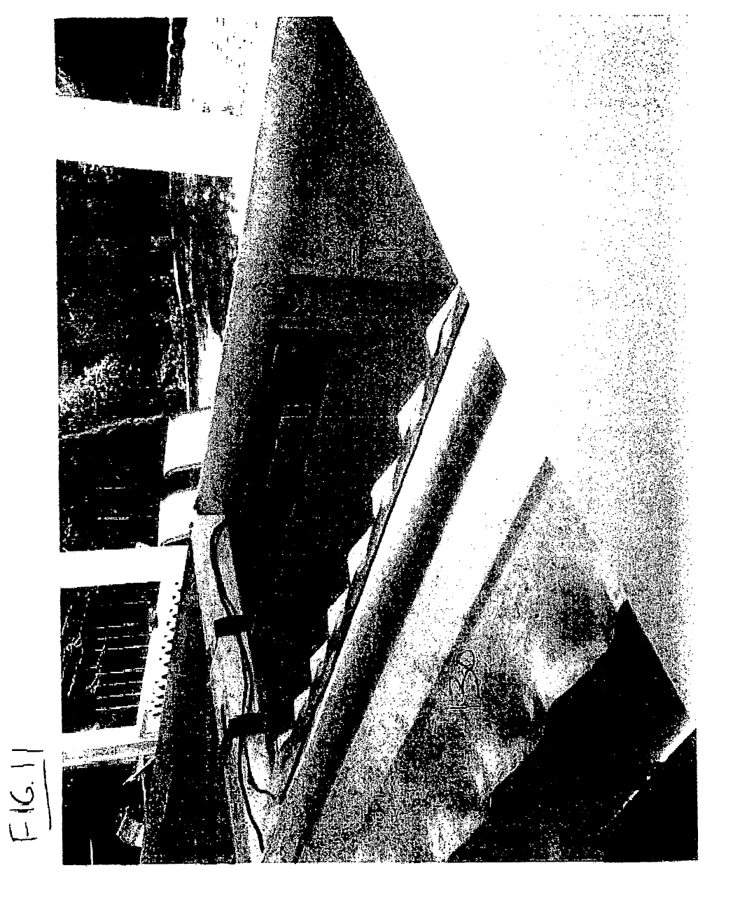

FIGS. 9-12 show various other embodiments of the present invention. It is noted that some embodiments have top panels 129, while some do not. Some have rails 140, while some do not. Thus, the present cart sanitizing system 120 can be individually tailored to some extent according to need and desire.

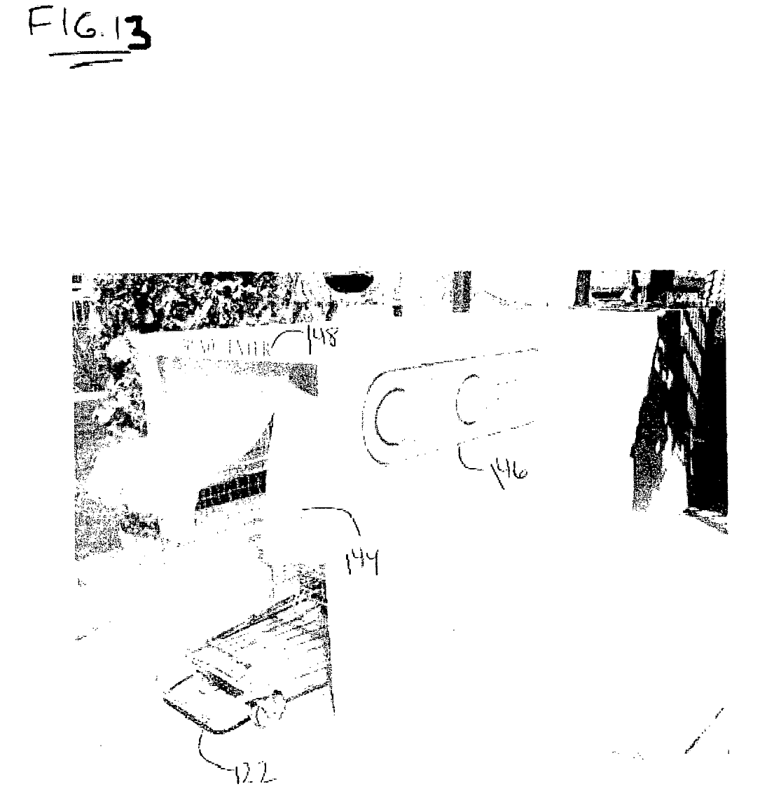

The embodiment shown in FIG. 13 also includes flaps 144, as well as an attractive logo 146, and other safety-related signs 148.

It is noted that the size and length of the enclosure 101 will depend on the number of carts 122 a given store will want to sanitize at one time. In some instances, as discussed below, perhaps only a single cart at a time needs to be sanitized, and as such a system with only single cart capacity would do. In contrast, in some situations, hundreds of carts may need to be sanitized per hour, thus necessitating a much larger system. However, no matter the size, the underlying principles remain largely the same.

The present system can be used inside or outside the store in the store's cart staging area.

In operation, the carts 122 are pushed or automatically feed though the enclosure 101 and are exposed to the UV or UVC light source 128 for a period of time. The time a cart is under the UV lamps will vary. For example, in one embodiment, the carts are exposed to the light source for 4 to 8 seconds. The amount of exposure time depends, in part, on how the system is manufactured. If it is desired that the carts are sanitized in less then 4 seconds, additional UV lamps can be added to the enclosure. In one embodiment, carts that are pushed straight through the enclosure will be irradiated for approximately 4-5 seconds. Through their exposure to the UV light, the carts are sanitized and disinfected. Once the carts have been sanitized, they can then be taken to a “clean area” where the customer can retrieve a clean germ free cart 122.

In the presently disclosed embodiments, the UV lamps are situated approximately ten inches from the top surface of the carts. Studies by the inventor have also demonstrated that UV lamps, emitting wavelengths of approximately 253.7 nm (nanometers) and placed approximately ten inches from a surface contaminated with strains of E. coli bacteria, are 100% effective in killing the E. coli.

It is also noted that while the proximity of the lamp in this embodiment is ten inches, it would be apparent to one skilled in the art to vary the proximity of the lamp to the cart surface, in order to have the desired germicidal/fungicidal effect. The necessary distance would depend on a variety of factors including, but not limited to, the intensity of the lamps, the amount of time the cart spends in the enclosure, and even the type of germs/viruses/fungi being treated.

One advantage to utilizing the UV light source 128 is that it is believed to kill all germs on the carts hard surfaces.

Variations of the Present Invention

It is understood that the above-described embodiments are only illustrative of the application of the basic principles of the present invention. Numerous modifications and alternative arrangements may be devised by those skilled in the art without departing from the spirit and scope of the present invention.

For example, in one embodiment, the switch 138 that turns on the UV light source is located on the rails 140. Thus, when a cart 122 is pushed into the enclosure 101, the wheels 142 activate the UV light source. By placing the switch 138 in this manner, it is no longer necessary for an individual to man the system 120, turning it on and off each time a cart 122 is deposited. Of course, it will be appreciated that a conventional light switch 138, as is depicted in FIG. 5, may not be best suited for placement on the rails 140. Rather, in this embodiment, a suitable type of switch would be a limit or in-line switch that once a cart reached a given point in the entrance of the enclosure it would turn the UV lamps on and would stay on until the enclosure was empty.

In yet another embodiment, the switch 138 is on the outside of the enclosure 101. Again, the specific type of switch would need to be tailored according to use. For instance, a suitable switching mechanism for this embodiment would be a lock or key switch. This switch could be turned on by a cart attendant and would activate the limit/line switches inside the enclosure. It is noted that these embodiments also help minimize the possibility that a child would enter the enclosure and trigger the UV light source.

In yet another embodiment, the UV light based sanitization system is an appendage to a conventional cart cleaning system (e.g. the carts could be washed with conventional cleaning solutions, and also sent through the UV light system).

With relatively little modification, the enclosure 101 could be kept outdoors, similar to conventional shopping cart return areas. Similarly, as discussed previously, the system could be modified such that it could be incorporated right into the building occupied by the retail vendor (i.e. the system could be free-standing, or alternatively, could be built into a larger structure, such as a building).

In yet another embodiment, the light source switch 138 requires a key. Thus, only authorized personnel can activate the switch 138.

In yet another embodiment of the present invention, the sanitizing system 120 is for single carts and is coin operated.

In yet another embodiment, the system is automated. For example, a cart is introduced into the system enclosure 101, which then switches on an automatic drive motor. The carts are then either pulled or pushed through the system enclosure 101 at slow speeds. Once the carts reaches a point in the enclosure 101 a series of special short wave UV lights come on and automatically turn off when the carts reach a second point. In this embodiment, the motor size and speed used depend on, among other things, how many carts the user wants to sanitize at a time.

In yet another embodiment, the system 100 can include a motion-activated switch 109 (FIG. 1) coupled to the UV light source (e.g. by an electric cord 111), and by which the UV light source is activated. The switching mechanism could be placed anywhere in the enclosure such that as carts enter the enclosure, their movement triggers the switch, thereby activating the UV light source. In yet other embodiments, the motion-activated switch could be further coupled to a timer, such that upon detecting the motion of the cart entering the enclosure, the UV light source is activated for a predetermined amount of time (e.g. 3-5 seconds). A wide number of motion-activated switch and timer configurations are commercially available, and their incorporation into the present invention would be apparent to one of ordinary skill in the art.

In the embodiments discussed above, the light source was typically taught as being on the top inside surface of the enclosure 101. However, it is considered within the scope of the present invention to have the UV light source 128 in any number of locations, or in multiple locations, throughout the enclosure 21 (e.g. on the side walls of the enclosure, etc.).

It is also noted that, in some instances, it may be desirable to increase the intensity of the light being emitted by the light source 128. For example, in some geographical areas, conditions may be more favorable for bacteria growth than in other areas. Thus, it may be desirable to irradiate a cart in those areas with higher intensity UV light. As would be apparent to one skilled in the art, the intensity of the UV radiation could be manipulated by simply increasing the power to the lamp (e.g. by selecting a different transformer, etc.).

What is claimed is:

1) A system for substantially disinfecting a cart against one or more contaminants, comprising:
a) an enclosure, adapted to receive a cart; and
b) a UV light source, coupled to the enclosure.

2) The system of claim 1, wherein the enclosure is a free-standing enclosure.

3) The system of claim 1, wherein the enclosure is integrated into a housing structure.

4) The system of claim 1, wherein the cart is one or more items selected from the group consisting of: shopping carts, warehouse carts, lumber carts and luggage carts.

5) The system of claim 1, wherein the UV light source is a cold cathode lamp.

6) The system of claim 1, wherein the UV light source is powered through a conventional power source.

7) The system of claim 1, wherein the UV light source is powered through an electric outlet.

8) The system of claim 3, wherein the UV light source is hard-wired into the housing structure.

9) The system of claim 1, wherein the UV light source is powered by a battery.

10) The system of claim 1, wherein the UV light source further includes a reflector.

11) The system of claim 6, further comprising a switch coupled between the power source and the UV light source.

12) A method of substantially disinfecting a cart against one or more contaminants, comprising the steps of:
a) providing an enclosure, adapted to receive the cart, and having a UV light source, coupled to the enclosure;
b) inserting the cart into the enclosure;
c) irradiating the cart with light emitted from the UV light source until a desired germicidal/fungicidal effect is obtained;
d) removing the cart from the enclosure, whereby the cart upon removal is substantially disinfected against the one or more contaminants.

13) The method of claim 12, wherein the enclosure is a free-standing enclosure.

14) The method of claim 13, wherein the enclosure is integrated into a housing structure.

15) The method of claim 13, wherein the cart is one or more items selected from the group consisting of: shopping carts, warehouse carts, lumber carts and luggage carts.

16) The method of claim 13, wherein the UV light source is a cold cathode lamp.

17) The method of claim 13, wherein the UV light source is powered through a conventional power source.

18) The method of claim 13, wherein the UV light source is powered through an electric outlet.

19) The method of claim 13, wherein the UV light source is powered by a battery.

20) The method of claim 13, wherein the UV light source further includes a reflector.
